(19) United States
(12) Patent Application Publication
(10) Pub. No.: US 2022/0202301 A1
(43) Pub. Date: Jun. 30, 2022

(54) METHOD FOR CHARACTERIZING BLOOD PRESSURE

(71) Applicant: Commissariat á l'Energie Atomique et aux Energies Alternatives, Paris (FR)

(72) Inventors: Mathilde LUBIN, Grenoble Cedex 09 (FR); Stéphane BONNET, Grenoble Cedex 09 (FR); Rémi GERBELOT BARILLON, Grenoble Cedex 09 (FR)

(73) Assignee: Commissariat á l'Energie Atomique et aux Energies Alternatives, Paris (FR)

(21) Appl. No.: 17/646,078

(22) Filed: Dec. 27, 2021

(30) Foreign Application Priority Data
Dec. 27, 2020 (FR) 20 14163

(51) Int. Cl. A61B 5/0225; A61B 5/00; A61B 5/021

(52) U.S. Cl. CPC A61B 5/02255; A61B 5/02141; A61B 5/7225

(57) ABSTRACT

A method for characterizing the blood pressure of an individual includes placing an optical device on a limb, the optical device having a light source arranged to emit light towards the limb, placing a photodetector facing the limb, and applying compression to the limb of the individual. The compression duration includes an increasing phase, in which the applied pressure increases, and a decreasing phase, in which the applied pressure decreases. During the compression duration, the limb is illuminated with the light source and the intensity of light is detected by the photodetector to obtain a time-dependent function. The method also includes applying low-pass filtering to the time-dependent function.

### TECHNICAL FIELD

The technical field is characterization of blood pressure using an optical method.

### PRIOR ART

Most devices allowing blood pressure to be characterized use a pressure sensor coupled to a compression cuff placed on a limb, generally an arm. Blood pressure is characterized by measuring the pressure exerted by the cuff at one or more characteristic times. The pressure sensor or acoustic sensor is sensitive to heartbeats and their amplitudes.

In mass-market blood-pressure monitors, a pressure sensor determines the air pressure in the cuff. The cuff is compressed so as to obtain arterial occlusion. During deflation of the cuff, pressure oscillations appear. The oscillations increase until they reach, transiently, a maximum amplitude. At this time, the pressure in the cuff is considered to be equal to the mean arterial pressure. From the detected maximum amplitude, the times respectively corresponding to the systolic and diastolic blood pressures are estimated on the basis of empirical laws. It is conventionally considered that:
the time corresponding to the systolic blood pressure is prior to the time of mean arterial pressure, and corresponds to an amplitude of oscillation of a certain percent of the maximum amplitude, 55% for example;
the time corresponding to the diastolic blood pressure is subsequent to the time of mean arterial pressure, and corresponds to an amplitude of oscillation of another percent of the maximum amplitude, 85% for example.

Such methods, which are conventionally employed in mass-market blood-pressure monitors, may be open to doubt, notably because of the use of empirical laws.

Optical measurements are frequently employed to determine physiological parameters of a living being. The technique of photoplethysmography (PPG) is a known technique, allowing variations in the volume of blood in blood vessels in living bodily tissues to be detected. Thus, PPG is a common technique for estimating heart rate.

Use of optical techniques has certain advantages, for example low cost and easy integration into compact devices intended for mass-market applications.

The inventors have developed an optical method that allows a characterization of blood pressure to be achieved.

### SUMMARY OF THE INVENTION

A first subject of the invention is a method for achieving characterization of a blood pressure of a user, comprising the following steps:
a) placing an optical device on a limb of the user, the optical device comprising:
   a light source, arranged to emit light towards the limb;
   a photodetector, the photodetector being arranged to detect an intensity of light emitted by the light source and having propagated through the limb;
b) applying compression to the limb of the individual, the compression being exerted between the heart of the user and the optical device, such that a pressure is applied to the limb for a compression duration, the compression duration comprising:
   an inflation phase, in which the applied pressure increases;
   a deflation phase, in which the applied pressure decreases;
c) during the compression duration, illuminating the limb with the light source and measuring an intensity detected by the photodetector at various times, so as to obtain a time-dependent function corresponding to a variation as a function of time in the intensity during the compression duration;
d) applying low-pass filtering to the time-dependent function, so as to extract a low-frequency component from the time-dependent function;
e) on the basis of the low-frequency component, characterizing the blood pressure of the individual.

The low-pass filtering is carried out with a cut-off frequency. Advantageously, the cut-off frequency is strictly lower than a heart rate of the individual. The cut-off frequency is for example lower than 1 Hz or than 0.5 Hz.

According to one embodiment, the characterization comprises estimating systolic blood pressure, the method comprising, during the inflation phase:
detecting a stabilization time beyond which the low-frequency component stabilizes and varies, following the stabilization time, notably in such a way as to form a plateau;
determining the pressure applied at the stabilization time.

By to form a plateau, what is meant is that the low-frequency component is considered to remain stable.

The method may comprise:
computing a derivative of the low-frequency component;
such that the stabilization time corresponds to the time at which, during inflation, the absolute value of the derivative drops below a stabilization threshold.

According to one embodiment, the characterization comprises estimating systolic blood pressure, the method comprising, during the deflation phase:
detecting an edge time at which the low-frequency component starts a phase of marked decrease following a phase of stability;
determining the pressure applied at the edge time.

By edge time at which the low-frequency component starts a phase of marked decrease following a phase of stability, what is meant is a time at which the variation as a function of time of the low-frequency component exhibits a sharp change in slope, passing from a low slope to a marked slope.

The method may comprise:
computing a derivative of the low-frequency component;
such that the edge time corresponds to the time at which, during deflation, the absolute value of the derivative rises above an edge threshold.

According to one embodiment, the characterization comprises estimating mean arterial pressure, the method comprising, during the deflation phase:
detecting a time at which the low-frequency component reaches a maximum slope;
determining the pressure applied at said time.

The method may comprise:
computing a derivative of the low-frequency component;
such that the time of estimation of the mean arterial pressure corresponds to a time at which, during deflation, the absolute value of the derivative is maximum.

According to one embodiment, the method comprises estimating a diastolic blood pressure from the systolic blood pressure and mean arterial pressure.

According to one embodiment, the characterization comprises estimating a time at which the low-frequency component reaches a minimum value, during deflation, this time corresponding to a time of venous return. The method may comprise determining the pressure applied at the time of venous return.

In step c), the limb may be illuminated in an illumination spectral band comprised between 500 nm and 1200 nm.

A second subject of the invention is an optical device for achieving characterization of a blood pressure, the device comprising:
a fastening, configured to fasten the optical device to a limb of a user;
a light source, configured to emit light that propagates towards the limb of the user when the device is being worn;
a photodetector, arranged to detect light emitted by the light source and having propagated through the limb of the user;
a control unit, programmed to implement steps d) to e) of a method according to the first subject of the invention, on the basis of the light detected by the photodetector.

Steps d) to e) are implemented during a compression of the limb. The compression may be achieved via a compression cuff, placed between the optical device and the heart of the user. The compression cuff may form part of the device or be associated with the device.

The invention will be better understood on reading the description of the examples of embodiment, which are described, in the rest of the description, with reference to the figures listed below.

### FIGURES

FIGS. 1A and 1B schematically show a first embodiment of a device according to the invention.

FIG. 1C shows another device embodiment according to the invention.

FIG. 2A shows a variation as a function of time in a pressure applied against a limb during a compression duration.

FIG. 2B shows a variation as a function of time in a pulse-related (i.e. high-frequency) component of the intensity of a light beam backscattered by the limb during the compression.

FIG. 3A shows a variation as a function of time in the pressure measured at a compression point, and a variation as a function of time in a low-frequency component of the intensity of a light beam backscattered by the limb, downstream of the compression.

FIG. 3B shows a variation as a function of time in a pulse-related component of the intensity of the light beam backscattered by the limb, during the trial described with reference to FIG. 3A.

FIG. 4 schematically shows the main steps of implementation of the invention.

### DESCRIPTION OF PARTICULAR EMBODIMENTS

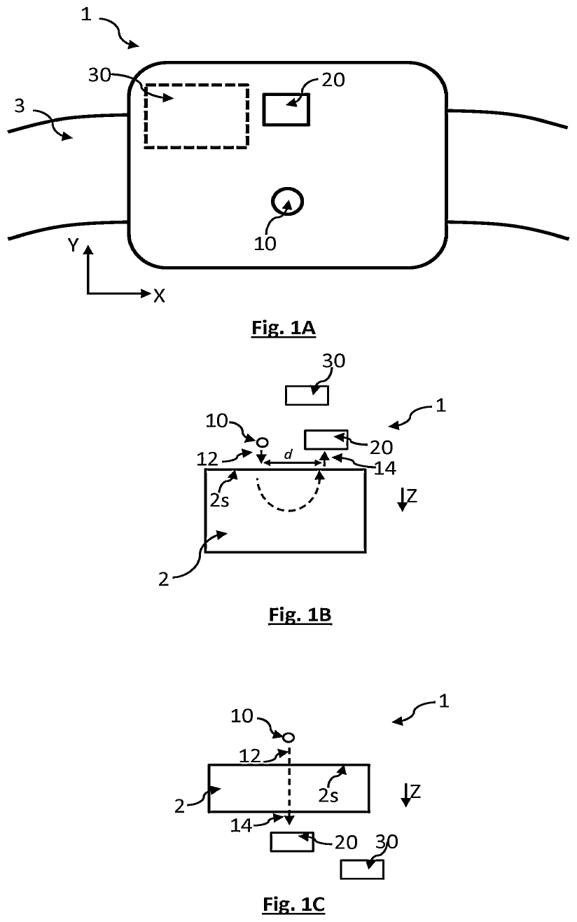

FIG. 1A shows an example of an optical device 1 allowing the invention to be implemented. The optical device 1 is intended to be applied against a limb 2 of a human or animal user. The limb 2 is for example an arm, a forearm, a wrist, a finger, or a leg. The limb against which the optical device is intended to be applied may be compressed, so as to temporarily block blood flow. In the example shown, the device 1 is connected to a fastening 3 forming a watch strap, so as to be fastened against a wrist. It may for example be integrated into a watch.

The device 1 comprises a light source 10, configured to emit a light beam towards the limb 2 facing which the light source is placed. The light source 10 emits an incident light beam 12 that propagates to the limb 2 along a propagation axis Z. The photons of the incident light beam 12 penetrate into the limb and some of said photons are backscattered, for example in a direction parallel to the propagation axis Z, back the way they came. The backscattered photons form backscattered radiation 14. The backscattered radiation 14 may be detected by a photodetector 20 placed facing a surface 2s of the limb. The photodetector 20 may be configured so as to detect backscattered radiation emanating from the sample at a distance d, called the backscatter distance, which is generally nonzero and smaller than a few millimetres, typically smaller than 15 mm or 10 mm. The photodetector 20 allows the intensity of the backscattered radiation to be measured.

FIG. 1B schematically shows the main components of the device 1 in a plane perpendicular to the surface 2s of the limb. The curved dashed arrow shows an optical path of photons emitted by the light source, in the analysed limb.

In the example shown, the light source emits in a spectral band centred on 660 nm, the bandwidth being of 10 nm. Preferably, the spectral band is comprised between 500 nm and 1200 nm, and more preferably between 600 nm and 1200 nm. In the following example, the source emits in a spectral band centred on 660 nm, the bandwidth being of 10 nm.

According to one possibility, the light source may comprise elementary light sources that emit in various spectral bands.

The optical device 1 comprises a processing unit 30 configured to process a signal detected by the photodetector 20. The processing unit 30 is connected to a memory, in which instructions for implementing the method described below are stored. The processing unit may comprise a microprocessor.

According to one alternative, shown in FIG. 1C, the limb 2 lies between the light source 10 and the photodetector 20. In such a configuration, which is called the transmission configuration, the photodetector 20 measures an intensity of photons having passed through the limb 2. Such a configuration assumes that the limb is sufficiently thin for a sufficient quantity of photons to emanate from the medium and to be detected by the photodetector.

Whatever the embodiment, the photodetector 20 is arranged to measure an intensity of a light beam formed by photons that have propagated through the limb 2: it is a question either of backscattered photons, or of photons having passed through the medium. In the rest of the description, the backscattering configuration shown in FIGS. 1A and 1B will be considered.

Implementation of the invention assumes that the limb 2, against which the device 1 is applied, is momentarily compressed, so as to decrease, or even block, blood flow through the portion of the limb against which the device 1 is placed. The compression is applied to the limb of the user upstream of a position of the device on the limb, the term upstream being to be considered with reference to arterial circulation. Thus, the compression is exerted between the heart of the user and the position of the device on the limb. In this example, the device is placed on the wrist of a person, the arm of whom may be compressed by a compression cuff. It may for example be a question of a cuff used in blood-pressure monitors such as described in the prior art. The cuff may be integrated into the device. The processing unit 30 is preferably connected to the cuff 3, in such a way that the processing unit receives information regarding whether the cuff is in an inflation or deflation phase.

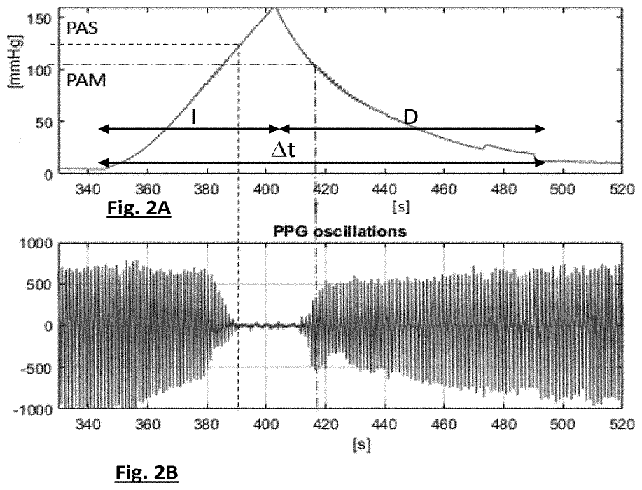

FIG. 2A shows a variation in a pressure P(t) applied against the limb 2 using the compression cuff. The x-axis corresponds to time. The y-axis represents the applied pressure. The cuff allows the limb to be compressed during a compression duration Δt, the latter possibly lasting a few tens of seconds, for example of the order of 1 minute. Conventionally, in the measurement of a blood pressure, the compression comprises an inflation phase I, in which the applied pressure increases, followed by a deflation phase D, in which the applied pressure decreases.

During deflation, and preferably also during inflation, the limb is illuminated by the light source 10, and the intensity of the backscattered beam 14, i.e. an intensity of photons emitted by the light source and having propagated through the examined limb, is measured. The intensity I(t) of the backscattered beam comprises a continuous, or low-frequency, component I_{DC}(t), and a pulse-related, or high-frequency, component I_{AC}(t), which is due to a periodic variation in the volume of the vessels under the effect of heartbeats. FIG. 2B shows the pulse-related component I_{AC}(t) of the intensity I(t). FIG. 2B also shows pressure variations measured, level with the cuff, by a pressure sensor. Under the effect of heartbeats, blood vessels located facing the light source are subjected to a periodic variation in their volume. Now, the intensity I(t) detected by the photodetector is an image of the variation in the amount of blood in the tissues facing the optical device 1. The higher the volume of blood, the greater the absorption and the lower the detected intensity. Thus, in the presence of a blood flow, the pulse-related intensity measured by the photodetector undergoes periodic variations at a frequency corresponding to the heart rate. In other words, the pulse-related component I_{AC}(t) contains oscillations representative of heartbeats.

When the pressure exerted by the cuff reaches and exceeds the systolic blood pressure (SP), blood flow is interrupted. The pulse-related component I_{AC}(t) then becomes negligible. After the pressure exerted by the cuff has led to the interruption of blood flow, the exerted pressure reaches a maximum supra-systolic level, then gradually decreases, until blood flow begins again. During deflation, when the pressure exerted by the cuff drops below the systolic pressure, the pulse-related component I_{AC}(t) once again exhibits oscillations. The amplitude of the oscillations increases, until a maximum, located at about 420 s in FIG. 2B, is reached. The pressure measured at this time is the mean arterial pressure (MAP).

In a way quite analogous to conventional measurements based on oscillations in cuff pressure, the pulse-related component allows a time at which the oscillations are maximum to be determined. At this time, the pressure of the cuff may be considered to correspond to the mean arterial pressure. The curves shown in FIGS. 2A and 2B show that analysis of the pulse-related component I_{AC}(t) allows the time at which the pressure exerted on the limb corresponds to the mean arterial pressure (MAP) to be estimated.

Use of a pulse-related component of a backscattered optical signal to estimate blood pressure is described in the publication Lubin M. et al “Blood pressure measurement by coupling an external pressure and photo-plethysmographic signals”, EMBC, July 2020. However, the inventors believe one drawback of this method to be that the pulse-related component I_{AC}(t) may be “drowned out” in variations in the continuous component I_{DC}(t). Thus, the extracted pulse-related component may be associated with a poor signal-to-noise ratio. In addition, the inventors have observed that the measurements of the pulse-related component are sensitive to the position of the device on the user.

The inventors have concluded that it is preferable to analyse the continuous component, i.e. a low-frequency component I_{DC}(t), rather than the pulse-related component I_{AC}(t) of the intensity detected by the sensor 20. This component is not used to estimate blood pressure. Specifically, the pulse-related component I_{AC}(t) is considered to contain more information, because of the similarities with the pressure oscillations measured during deflation of a cuff.

Thus, and it is an important element of the invention, the “low-frequency” intensity I_{DC}(t) of the beam 14, once “disencumbered” of the pulse-related component, turns out to be a relevant indicator, allowing an estimation of the time at which the pressure exerted by the cuff corresponds to the systolic blood pressure or to the mean arterial pressure or to another characteristic blood pressure.

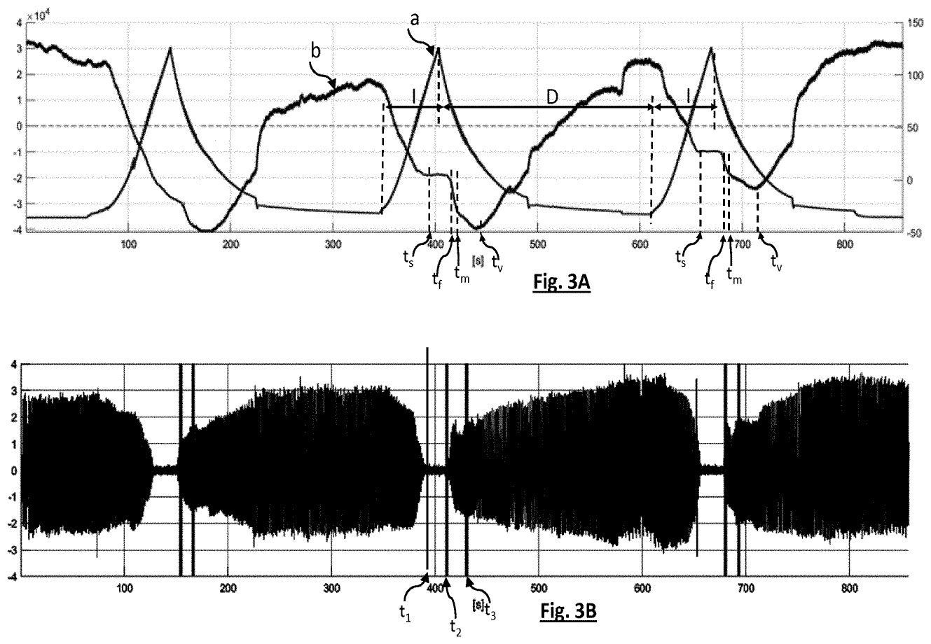

FIG. 3A shows the pressure exerted by the cuff on the arm of the user (curve a—y-axis to the right—arbitrary units) as a function of time (x-axis—unit seconds). A plurality of inflation I/deflation D cycles have been shown in FIG. 3A. In the course of each inflation and of each deflation, the arm is illuminated by the light source 10 and the photodetector 20 measures an intensity of backscattered light. The intensity I(t) of the backscattered light is measured at a sampling frequency that is preferably higher than 10 Hz, or even higher than 100 Hz. The higher the sampling frequency, the better the time-domain resolution of the measurements. Thus, a time-dependent variation I(t) in intensity is obtained during the compression duration Δt. The measured time-dependent variation I(t) is subjected to low-pass filtering, so as to preserve only frequency components lower than a cut-off frequency. Thus, the low-frequency component I_{DC}(t) is obtained. In order to remove the periodic component described with reference to FIG. 2B, the cut-off frequency is strictly lower than the heart rate of the user. The cut-off frequency is generally lower than 1 Hz. It may be comprised between 0.1 Hz and 1 Hz. It may for example be a few 0.1 Hz, for example 0.5 Hz. FIG. 3A shows a variation in the low-frequency component I_{DC}(t) during each inflation/deflation cycle: see curve (b)—y-axis to the left—arbitrary units.

The low-frequency component I_{DC}(t) reflects the quantity of blood present in the tissues located facing the optical device 1. During the inflation, blood flow decreases little by little, until blocked. The accumulation of blood leads to a decrease in the backscattered signal, and therefore in the low-frequency component I_{DC}(t) because of an increase in the absorption of photons by the blood accumulating downstream of the compression. When blood flow is blocked, the low-frequency component stabilizes, thereby forming a plateau. This plateau corresponds to a relative stagnation of the intensity I_{DC}(t). The plateau extends between:
a stabilization time tₛ that corresponds to the time at which the pressure exerted by the cuff, during inflation, reaches then exceeds the systolic blood pressure SP;
an edge time t_{f}, subsequent to the stabilization time, that corresponds to the time at which the pressure exerted by the cuff, during deflation, reaches the systolic blood pressure and drops below the latter.

Between the start of the inflation and the stabilization time tₛ, the derivative I′_{DC}(t) of I_{DC}(t) is negative, this expressing a decrease in I_{DC}(t). The stabilization time t₀ corresponds to the time at which, during inflation, the derivative I′_{DC}(t) drops to zero, or, more generally, the absolute value |I_{DC}(t)| of the derivative I′_{DC}(t) drops below a threshold, called the stabilization threshold Thₛ. The stabilization time corresponds to a time at which, during inflation, I_{DC}(t) passes from a marked decrease to a moderate decrease, or even a stabilization.

Thus, at tₛ,

$$\vert {I}_{DC}^{′} ( {t}_{s} ) \vert \leq {Th}_{s} . \qquad ( 1 )$$

The edge time t_{f} corresponds to a time at which, during deflation, the derivative I′_{DC}(t) of the low-frequency component is negative, and the absolute value of the derivative |I′_{DC}(t)| rises above an edge threshold Th_{f}. The edge threshold may have the same value as the stabilization threshold mentioned in the preceding paragraph. The edge time corresponds to a time at which, during deflation, I_{DC}(t) passes from relative stability to a marked decrease, forming an edge.

Thus, at t_{f},

$$\vert {I}_{DC}^{′} ( {t}_{f} ) \vert \geq {Th}_{f} \qquad ( 2 )$$

Between the stabilization time tₛ and the edge time t_{f}, the cuff exerts a pressure higher than the systolic blood pressure: the pressure exerted is said to be supra-systolic.

During deflation, after the edge time t_{f}, blood flow starts again, firstly through the arteries. However, the veins, which are more supple than the arteries, remain occluded temporarily by the pressure exerted by the cuff. An additional accumulation of blood results thereby, this resulting in a substantial decrease in the intensity of the backscattered signal. The detected signal reaches a maximum slope, at a time tₘ of maximum slope. The time tₘ is considered to be the time at which the slope of I_{DC}(t) is maximum during deflation. This corresponds to a maximum value of |I′_{DC}(t)| during deflation. At this time, the pressure exerted by the cuff is considered to correspond to the mean arterial pressure MAP: the artery is said to be compliant.

During deflation, when the venous occlusion ceases, this being designated by the term “venous return”, the amount of blood facing the device decreases. Absorption of photons by the blood also decreases, this leading to an increase in detected intensity. This corresponds to the time at which, during deflation, the derivative I′_{DC}(t) becomes positive again. The time of venous return is denoted tᵥ. It corresponds to the time at which I_{DC}(t) is minimum. It is then possible to measure the pressure of the cuff at the time tᵥ of venous return.

In FIG. 3B, the pulse-related component I_{AC}(t) of the detected signal (y-axis—arbitrary units) has been shown as a function of time (x-axis—unit seconds—same time axis as FIG. 3A). Times t₁ and t₂ that correspond to stoppage of the oscillations during inflation and to restart of the oscillations during deflation, respectively, may be seen. These times correspond to the times tₛ and t_{f} described with reference to FIG. 3A, respectively. A time t₃ that corresponds to the diastolic pressure has also been shown in FIG. 3B. The times t₁, t₂ and t₃ were determined by a medical doctor.

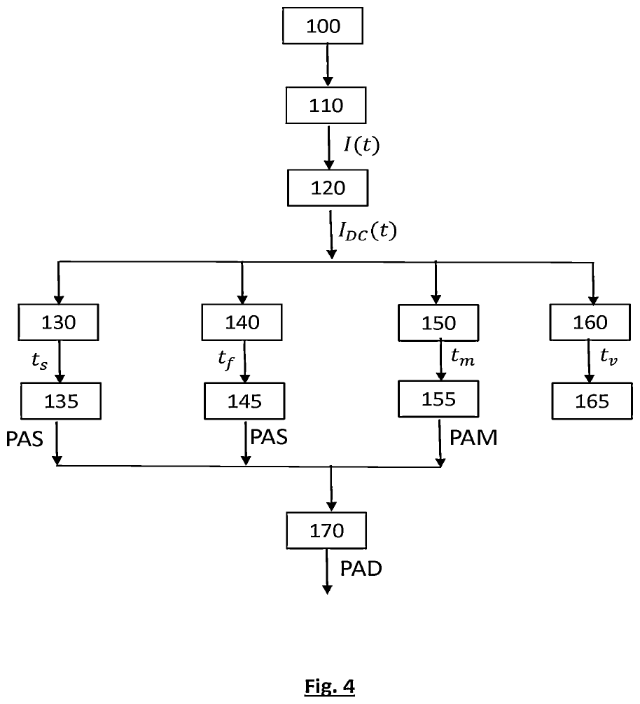

FIG. 4 schematically shows the main steps of a method according to the invention, which are described below:

Step 100: Applying a pressure to the limb 2, upstream of the optical device, during a compression duration. The term upstream is to be understood with reference to the direction of arterial flow. The compression duration comprises an inflation phase I, in which the applied pressure increases, and a deflation phase D, in which the applied pressure decreases.
Step 110: Measuring the intensity of the light backscattered or transmitted by the limb 2 during the compression. Thus, a time-dependent function representing a variation in the intensity 40 during compression is obtained.
Step 120: Low-pass filtering of the function I(t), the cut-off frequency of the filter being strictly lower than the heart rate of the user. Thus, the low-frequency component I_{DC}(t) of the measured intensity, or low-frequency intensity, is obtained. The filtering may be obtained using a moving average, a Gaussian filter, or a more refined low-pass filter.
Steps 130, 135 or 140, 145, which are described below, aim to determine the systolic blood pressure. Either steps 130 and 135, or steps 140 and 145, or all of these steps, are implemented. When all of these steps are implemented, two estimations of systolic pressure are obtained, which may be averaged, or only the estimation considered to be most reliable of which is retained.
Step 130: Detecting, during inflation, the stabilization time tₛ, at which the decreasing low-frequency component I_{DC}(t) reaches a plateau, or, more generally, may be considered to remain stable. The stabilization time may be detected as corresponding to a time at which, during inflation, the absolute value |I′_{DC} (t)| of the derivative I′_{DC}(t) drops below the stabilization threshold Thₛ, this threshold possibly for example being 0 or close to 0. The threshold Thₛ may be preset.
Step 135: Measuring the pressure applied at the stabilization time tₛ resulting from step 130. The measured pressure corresponds to the systolic blood pressure SP. In other words, SP=P(tₛ).
Step 140: During the deflation phase, detecting the edge time t_{f}, which corresponds to the time at which the systolic pressure is reached. The edge time corresponds to a time at which, during deflation, the absolute value |I′D_{c}(t)| of the derivative I′_{DC}(t) rises above the edge threshold Th_{f}. The threshold Th_{f} may be preset.
Step 145: Measuring the pressure applied at the edge time t_{f}. The measured pressure corresponds to the systolic blood pressure SP. In other words, SP=P(t_{f}).
Step 150: During the deflation phase, detecting the time tₘ at which the slope of I_{DC}(t) is maximum. This time corresponds to the time at which the mean arterial pressure is reached.
Step 155: Measuring the pressure applied at the time tₘ. The measured pressure corresponds to the mean arterial pressure MAP. In other words, MAP=P(tₘ).
Step 160: During the deflation phase, detecting the time tᵥ at which the value I_{DC}(t) is minimum. This time corresponds to the time of venous return.
Step 165: Measuring the pressure at the time tᵥ.
Step 170: In this step, on the basis of the MAP resulting from step 155, and of the SP resulting from step 135 and/or from step 145, the diastolic blood pressure is determined, using an empirical relationship:

$$D P = \frac{3}{2} M A P - \frac{1}{2} S P \qquad ( 3 )$$

Steps 130/135, 140/145, 150/155 and 160/165 may be implemented independently of one another. The method may comprise all these steps, or only some of these steps. For example, if only the MAP is sought, only steps 150 and 155 are implemented after step 120.

The invention allows a reliable characterization of blood pressure, using simple optical components that may be integrated into portable devices, worn by the user: it may for example be a question of instrumented watches or armbands.

1. A method for characterizing a blood pressure of an individual, comprising:
a) placing an optical device on a limb of the individual, the optical device comprising:
a light source, arranged to emit light towards the limb; and
a photodetector, the photodetector being arranged to detect an intensity of light emitted by the light source and having propagated through the limb;
b) applying compression to the limb of the individual, the compression being exerted between the heart of the individual and the optical device, such that a pressure is applied to the limb during a compression duration, the compression duration comprising:
an inflation phase, in which the applied pressure increases; and
a deflation phase, in which the applied pressure decreases;
c) during the compression duration, illuminating the limb with the light source and measuring an intensity detected by the photodetector at various times, so as to obtain a time-dependent function corresponding to a variation as a function of time in the detected intensity during the compression duration;
d) applying a low-pass filter to the time-dependent function, so as to extract a low-frequency component from the time-dependent function, the low-pass filtering being carried out with a cut-off frequency lower than a heart rate of the individual; and
e) on the basis of the low-frequency component, characterizing the blood pressure of the individual.

2. The method of claim 1, wherein the cut-off frequency is lower than 1 Hz.

3. The method of claim 1, wherein e) comprises:
using the low-frequency component to determine at least one characteristic time; and
determining the blood pressure from the applied pressure at said characteristic time.

4. The method according to claim 1, wherein the characterization comprises estimating systolic blood pressure, the method comprising, during the inflation phase:
detecting a stabilization time beyond which the low-frequency component stabilizes and varies, following the stabilization time, in such a way as to form a plateau; and
determining the pressure applied at the stabilization time.

5. The method according to claim 4, comprising:
computing a derivative of the low-frequency component, wherein the stabilization time corresponds to a time at which, during the inflation phase, an absolute value of the derivative drops below a stabilization threshold.

6. The method according to claim 1, wherein characterizing the blood comprises estimating systolic blood pressure, the method comprising, during the deflation phase:
detecting an edge time at which the low-frequency component starts a phase of marked decrease following a phase of stability; and
determining the pressure applied at the edge time.

7. The method according to claim 6, comprising:
computing a derivative of the low-frequency component, wherein the edge time corresponds to a time at which, during the deflation phase, an absolute value of the derivative rises above an edge threshold.

8. The method according to claim 1, wherein characterizing the blood comprises estimating a mean arterial pressure, the method comprising, during the deflation phase:
detecting a time at which the low-frequency component reaches a maximum slope; and
determining the pressure applied at the detected time.

9. The method according to claim 8, comprising:
computing a derivative of the low-frequency component, wherein a time of estimation of the mean arterial pressure corresponds to a time at which, during the deflation phase, an absolute value of the derivative is maximum.

10. The method according to claim 4, wherein characterizing the blood comprises estimating a mean arterial pressure, the method comprising:
estimating a diastolic blood pressure from the systolic blood pressure and mean arterial pressure.

11. The method according to claim 1, wherein characterizing the blood comprises estimating a time at which the low-frequency component reaches a minimum value, during the deflation phase, the estimated time corresponding to a venous return time.

12: The method according to claim 11, comprising determining the pressure applied at the venous return time.

13: The method according to claim 1, wherein, in c), the limb is illuminated in an illumination spectral band comprised between 500 nm and 1200 nm.

14: An optical device for characterizing a blood pressure,
the device comprising:
a fastening configured to fasten the optical device to a limb of an individual;
a light source configured to emit light that propagates towards the limb of the individual when the device is being worn;
a photodetector arranged to detect light emitted by the light source and having propagated through the limb of the individual; and
a control unit, configured to implement steps d) to e) according to the method of claim 1, on a basis of the light detected by the photodetector.

15: The method of claim 1, wherein the cut-off frequency is lower than 0.5 Hz.
